(19) United States
(12) Patent Application Publication
(10) Pub. No.: US 2007/0020670 A1
(43) Pub. Date: Jan. 25, 2007

(54) Methods for detecting and confirming minimal disease

(75) Inventors: Michael R. Loken, Mercer Island, WA (US); Barbara K. Zehentner-Wilkinson, Poulsbo, WA (US)

Correspondence Address: SEED INTELLECTUAL PROPERTY LAW GROUP PLLC, 701 FIFTH AVE, SUITE 5400, SEATTLE, WA, 98104, US

(73) Assignee: HematoLogics, Inc., Seattle, WA (US)

(21) Appl. No.: 11/484,004

(22) Filed: Jul. 7, 2006

Related U.S. Application Data
(60) Provisional application No. 60/697,694, filed on Jul. 7, 2005.

(51) Int. Cl. C12Q 1/68; C12P 19/34

(52) U.S. Cl. 435/6; 435/91.2

(57) ABSTRACT

The present invention provides improved methods for detection of minimal disease. More specifically, the invention provides methods for combining cell sorting with clonality profiling to effectively lower sensitivity limits for disease detection and to provide independent confirmation of the tumor detection without the need for patient specific assay designs.

### BACKGROUND OF THE INVENTION

1. Field of the Invention

The present invention relates generally to improved methods for confirming the presence of minimal disease in cancer patients and, more particularly, to methods useful for initial diagnostics and for monitoring the presence of minimal residual disease following treatment.

2. Description of the Related Art

The detection of minimal disease can play a significant role not only in monitoring response to therapy but also in the accurate diagnosis of the underlying cause of major clinical signs. Several studies have shown that quantitative detection of minimal residual disease (MRD) in lymphoid malignancies predicts clinical outcome. (Szczepanski T, et al., Lancet Oncol 2001; 2: 409-17; van Dongen J J, et al., Lancet 1998; 352: 1731-8; Bruggemann M, et al., Acta Haematol 2004; 112: 111-9; Cave H, et al., N Engl J Med 1998; 339: 591-8; Coustan-Smith E, et al., Blood 2000; 96: 2691-6; Coustan-Smith E, et al., Blood 2002; 100: 52-8; Wells DA, et al., Am J Clin Pathol 1998; 110: 84-94; Radich J, et al., Biol Blood Marrow Transplant 1995; 1:24-31; Bahloul M, et al., Best Pract Res Clin Haematol 2005; 18: 97-111; Hoshino A, et al., Tohoku J Exp Med. 2004; 203: 155-64; Ciudad J, et al., Br J Haematol 1999; 104: 695-705; Lucio P, et al., Leukemia 1999; 13: 419-27.) Monitoring treatment response by tumor load quantification is crucial to assess risk of relapse and for determining those patients who may benefit from therapy reduction, intensification, reduction of immunosuppression for graft versus leukemia effect post stem cell transplant, or adoptive T cell therapy. (Bradfield SM, et al., Leukemia 2004; 18: 1156-8.) Minimal disease may also be encountered in diagnostic situations. For example, low levels of monoclonal B cells in patients with cytopenias suspicious for myelodysplastic syndromes. (Wells DA, et al., Blood 2003; 102: 394-403.) Minimal disease detection is also encountered in staging of lymphoma which may require the detection of low levels of tumor in a background of normal cells. Thus, detection of minimal disease is not limited to monitoring treatment but can be necessary in diagnostic settings where no reference population is available for comparison.

Immunoglobulin (Ig) and T cell receptor (TCR) gene rearrangements are frequently used as targets in PCR-based MRD studies. (van der Velden VH, et al., Leukemia 2003; 17: 1013-34; van der Velden VH, et al., Leukemia 2002; 16: 1372-80; van der Velden VH, et al., Leukemia 2002; 16: 928-36.) These rearrangements can be considered as ‘fingerprints’ for lymphoid cells since each clone has its own deletions and random insertion of nucleotides at the junction sites of the gene segments. A clonal leukemic cell population of lymphoid origin can be detected by the presence of a strong signal for a single gene rearrangement of a specific size after multiplex PCR amplification followed by fluorescence based capillary electrophoresis whereas a polyclonal lymphocyte population results in uniform Gaussian distribution of amplicons. In order to use these gene rearrangements for MRD analysis by real-time PCR, patient specific gene probes must be created by sequencing the gene rearrangement amplicon, designing primers and optimizing assay sensitivity. In addition to the cumbersome set-up of patient specific assays, several gene rearrangements generally must be used simultaneously as PCR targets since single rearrangements are unstable and can be lost during clonal transformation and following disease relapse due to continuing gene rearrangements or further gene deletions. In particular for malignancies demonstrating oligoclonality with multiple subclones present at diagnosis, the likelihood of losing a PCR-target during follow-up is significantly increased. (Beishuizen A, et al., Blood 1994; 83: 223847.)

Thus, conventional detection of residual disease using patient-specific reagent panels suffers from the following limitations:

1. A diagnostic specimen with an aberrant phenotype is required in order to construct a panel. In 25% of cases an aberrant phenotype may not be identifiable. (San Miguel J F, et al., Blood 2002; 98: 1746-1751.). .). Note that the specimen may also not be available as the patient may have been diagnosed and treated elsewhere and no sample was saved.

2. Processing time is substantial because a technician must examine prior analysis for the particular patient in order to determine the reagent combination to use in each case.

3. The phenotype of a leukemic cell population that is different than the originally diagnosed phenotype may not be detected. For example, the phenotype may change from diagnosis to relapse as a result of clonal evolution or an outgrowth of a minor chemotherapy resistant subclone. (See San Miguel, supra.)

4. Unexpected or unanticipated abnormalities, such as secondary myelodysplasia or abnormalities in other lineages may be overlooked.

The assessment of residual disease using patient-specific panels can work well in a controlled environment, such as a research study where there is access to all sequential specimens and there is high compliance in obtaining specimens at specific times in therapy. In clinical practice, however, a flow cytometry laboratory may be asked to perform residual disease analysis when the laboratory did not perform the initial diagnosis. A detailed immunophenotype is often unavailable or incomplete. Thus, there are cases where minimal disease must be detected without a prior diagnostic specimen. Either the specimen is not available or the tumor is detected at low levels as a primary diagnosis.

Accordingly, there remains a need in the art for improved methods for quickly confirming the presence of minimal disease in cancer patients without the need for patient-specific reagents, particularly methods using small numbers of rare cells. The present invention fulfills this need and other needs.

### BRIEF SUMMARY OF THE INVENTION

On aspect of the present invention provides a method for detecting the presence of minimal disease in a cancer patient, comprising, identifying a population of abnormal cells by flow cytometry; sorting the population of abnormal cells; and contacting nucleic acid isolated from the sorted cells with one or more oligonucleotides, wherein the one or more oligonucleotides are not patient-specific, and wherein the contacting determines the presence of a neoplastic genetic marker; thereby detecting the presence of minimal disease. In one embodiment, the step of identifying the population of abnormal cells by flow cytometry comprises measuring forward scatter and side scatter in combination with the fluorescence intensity of a combination of two or more cell surface markers selected from CD10, CD45, CD19, CD34, CD20, CD22, CD45, CD3, CD56, CD4, CD8, CD5, CD7, and CD2. In further embodiments, the two or more cell surface markers comprise CD5 and CD19, CD5 and CD8, CD10 and CD20, CD3 and CD56, CD3 and CD4, CD3 and CD8, CD5 and CD7, CD5and CD3, CD2 and CD7, CD2 and CD3, CD5 and CD2, CD38 and CD56, CD138 and CD38, CD138 and CD19, or CD38 and CD19.

In an additional embodiment, the nucleic acid is contacted with at least two oligonucleotides in a polymerase chain reaction. In a further embodiment, the at least two oligonucleotides specifically amplify clonally rearranged immunoglobulin genes. In this regard, illustrative clonally rearranged immunologlobulin genes include, but are not limited to Ig heavy chain rearrangements, Ig kappa gene rearrangements, and Ig lambda gene rearrangements. In a further embodiment, the at least two oligonucleotides specifically amplify clonally rearranged T cell receptor genes. In this regard, illustrative clonally rearranged T cell receptor genes include but are not limited to T cell receptor beta chain gene rearrangements, T cell receptor delta chain gene rearrangements, and T cell receptor gamma chain gene rearrangements. Thus, in certain embodiments, the neoplastic genetic marker is a clonally rearranged T cell receptor gene and/ or a clonally rearranged immunoglobulin gene.

In one embodiment of the methods described herein, the number of sorted cells is between about 200 and 1000. In another embodiment, the presence of minimal disease in the cancer patient is confirmed in about 2 days, in about 3 days, or in about 4 days. In another embodiment of the method, the nucleic acid is DNA or RNA. In certain embodiments, the minimal disease is minimal residual disease. In a further embodiment, the population of abnormal cells comprises neoplastic B cells present at between about 0.8% and 0.001% of nucleated cells. In another embodiment, the population of abnormal cells comprises neoplastic T cells present at between about 0.8% and 0.001% of nucleated cells.

Another aspect of the present invention provides a method for detecting the presence of minimal disease in a cancer patient, comprising, identifying a population of abnormal cells by flow cytometry; sorting the population of abnormal cells; and contacting nucleic acid isolated from the sorted cells with at least two oligonucleotides in a polymerase chain reaction, wherein the at least two oligonucleotides specifically amplify clonally rearranged immunoglobulin genes and are not patient-specific; and wherein the amplification of a clonal population confirms the presence of minimal disease.

A further aspect of the present invention provides methods for detecting the presence of minimal disease in a cancer patient, comprising, identifying a population of abnormal cells by flow cytometry; sorting the population of abnormal cells; and contacting nucleic acid isolated from the sorted cells with at least two oligonucleotides in a polymerase chain reaction wherein the at least two oligonucleotides specifically amplify clonally rearranged T cell receptor genes and are not patient-specific; and wherein the amplification of a clonal population confirms the presence of minimal disease.

Another aspect of the present invention provides a method for detecting the presence or absence of minimal disease in a cancer patient, comprising, identifying a population of cells suspected of containing abnormal cells by flow cytometry; enriching the population of cells suspected of containing abnormal cells by sorting said population of cells; and contacting nucleic acid isolated from the enriched, sorted cells with one or more oligonucleotides, wherein the one or more oligonucleotides are not patient-specific, and wherein the contacting determines the presence or absence of a neoplastic genetic marker; thereby detecting the presence or absence of minimal disease. In one embodiment of the methods, the population of cells suspected of containing abnormal cells comprises plasma cells. In a further embodiment of the method, the neoplastic genetic marker is a clonally rearranged immunoglobulin gene. In an additional embodiment, the nucleic acid is contacted with at least two oligonucleotides in a polymerase chain reaction. In a further embodiment, the at least two oligonucleotides specifically amplify clonally rearranged immunoglobulin genes. In yet a further embodiment, the clonally rearranged immunologlobulin gene is selected from the group consisting of an Ig heavy chain rearrangement, an Ig kappa gene rearrangement, and an Ig lambda gene rearrangement.

These and other aspects of the present invention will become apparent upon reference to the following detailed description and attached drawings.

### BRIEF DESCRIPTION OF THE DRAWINGS

FIG. 1: Bone marrow aspirate from a patient with a diagnosis of follicular center cell lymphoma was analyzed by multidimensional flow cytometry for staging. A: CD45 gating was used to distinguish between immature cells (Blast), mature lymphocytes (Lymph), monocytes (Mono) and maturing myeloid cells (Myeloid). B: A slight increase in CD20+/CD10+cells were detected in the cells confined to the Lymph+Blast gate (A). C,D: Analysis of the light chain restriction on the mature B lymphoid cells showed a small population of dim CD19+cells that expressed predominantly lambda. E,F: Immunoglobulin light chain analysis on the CD10+cells also shows an increase in lambda positive cells.

FIG. 2: B cell gene rearrangement analysis of genomic DNA specimens derived from a staging lymphoma bone marrow specimen with 0.6% phenotypically abnormal lymphocytes (FIG. 1). Unsorted bone marrow (a): Monoclonal peaks detected among polyclonal background at 346 bp for immunoglobulin heavy chain framework region one (blue) and at 281 bp for framework region two (black). IGH FR3 (green) shows polyclonal amplicon distribution only. CD10 positive sorted cell fraction (b): Monoclonal amplicons detected with identical sizes to the unsorted bone marrow specimen for IGH FR2 (black) and FR1 (blue). CD10 negative sorted control cell fraction (c): Polyclonal amplicon distribution for all three framework regions.

FIG. 3: Bone marrow from a patient with precursor B acute lymphoblastic leukemia after re-induction therapy following relapse after hematopoeitic stem cell transplant. CD45 gating was used to identify the blasts (red) and mature lymphocytes (blue) as described in FIG. 1A. A small population of cells (0.05%) that expressed CD19 and CD10 but not CD45 or CD20 was detected (Red dots), A, C, E. Analysis 5 weeks later showed an increase in the same abnormal population (4.8%, red) but now combined with normal developing B lymphoid precursors and T cells (blue), B, D, F. Cells within the red parallelogram, C, were sorted for IgH gene rearrangement studies.

FIG. 4: B cell gene rearrangement analysis of follow-up bone marrow specimens from a patient with precursor B acute lymphoblastic leukemia after re-induction therapy following relapse after hematopoeitic stem cell transplant. (a) The sorted tumor cell population (0.05% abnormal lymphoblasts, FIG. 3C) had a monoclonal peak profile with amplicons at 115 bp and 164 bp for IgH framework region three (FR3, green), 254 bp for FR2 (black) and 314 bp and 360 bp for FR1 (blue). No monoclonal or polyclonal signal was detected for the corresponding unseparated bone marrow specimen. (b) Unseparated follow-up bone marrow specimen five weeks later with 4.8% abnormal lymphoblasts (FIG. 3B,D,F) resulted in IgH clonality profile with identical amplicon sizes to previous specimen. (c) Unseparated bone marrow specimen 4 weeks later after further chemotherapy. B cell gene rearrangement analysis did not result in a distinct monoclonal profile due to the low percentage of tumor cells. (d) B cell clonality profile identical to previous amplicon sizes detected in the residual abnormal lymphoblast population (0.3% of total nucleated cells) sorted by flow cytometry.

FIG. 5: Bone marrow aspirate from a patient with hemolytic anemia and cryoglobulin revealed a small population (0.6%) of mature B lymphoid cells (gated as Lymph, FIG. 1A) that co-expressed CD5 and CD19, A. Four color analysis combining CD45 bright, CD19+, CD5+ and immunoglobulin light chain demonstrates an increase in kappa relative to lambda staining on this minor cell population, B,C. Total B lymphoid cells in this specimen (CD19+, bright CD45, without expression of CD5, (blue rectangle A) were enumerated at 6.1 %. The CD5+,CD19+ (A) cells were sorted for IgH gene rearrangement studies (FIG. 6).

FIG. 6: B cell gene rearrangement analysis of the bone marrow specimen from a patient with hemolytic anemia and cryoglobulin (FIG. 5). (a) A suspicious but not definitive monoclonal peak was detected at 320 bp for the immunoglobulin heavy chain framework one region (blue) in the unseparated bone marrow. (b) The abnormal B-lymphoid population detected by flow cytometry at 0.6% (FIG. 5A) was sorted by CD5 and CD19 positivity and analyzed for B-cell gene rearrangements. A distinct monoclonal peak at 320 bp for FR1 was detected in the purified tumor cell fraction.

FIG. 7: Peripheral blood from a patient with anemia demonstrated a homogeneous T cell population expressing increased CD3 and CD56 at 5% of total nucleated cells or 11% of mature lymphocytes. The populations in the oval and in the rectangle were sorted for TCR gene rearrangement studies.

FIG. 8: Total peripheral blood from a patient with anemia was analyzed by T cell receptor gamma gene rearrangement PCR and a putative monoclonal peak was detected at 245 bp (a). Using a CD56+ and CD3+ gate, the abnormal T cell population (FIG. 7) was purified and a single monoclonal peak at 245 bp was detected by TCRG analysis. (b) As an internal control the normal T cells, not expressing CD56, was isolated by flow cytometry based cell sorting and subsequent TCRG analysis detected no distinct clonal peak.

### DETAILED DESCRIPTION OF THE INVENTION

The present invention relates generally to improved methods for confirming the presence of minimal disease in cancer patients as well as to methods useful for initial diagnostics and for monitoring the presence of minimal residual disease following treatment.

Flow cytometric-based immunophenotyping provides a rapid and sensitive method for detecting up to one leukemic cell in 10⁴ normal cells. Molecular analysis of gene rearrangements can routinely detect a minimum of 1 monoclonal B cell in 1000 normal cells using immunoglobulin (Ig) heavy chain multiplex assays and 1 monoclonal T cell in 100 normal cells using T cell receptor (TCR) gamma primer sets. Patient specific real-time quantitative PCR (RQ-PCR) assays can be established for Ig/TCR gene rearrangements with sensitivities ranging from 0.01% up to 0.001%, but assay set-up is currently too time-consuming for a routine clinical application. In addition, access to a diagnostic specimen is necessary in order to develop a patient specific RQ-PCR assay.

There are cases where minimal disease must be detected without a prior diagnostic specimen. Either the specimen is not available or the tumor is detected at low levels as a primary diagnosis. By using the combination of molecular and flow cytometric cell sorting techniques it is possible to have better sensitivity and confirmed specificity by merging phenotypic with genotypic analysis. The present invention provides a quantitative two-step technique for detecting and confirming low levels of disease by integrating phenotype analysis using standardized flow cytometry panels, cell sorting and genotype analysis using multiplex gene rearrangement PCR thereby confirming the presence of both aberrant phenotype linked to a specific genotype.

Fluorescence Activated Cell Sorting

Gene products can be identified on the cell surface or in the cytoplasm of cells using specific monoclonal antibodies. Flow cytometry can be used to detect multiple immunofluorescent markers simultaneously in a quantitative manner. The technique of immunofluorescent staining is well known and can be carried out according to any of a variety of protocols, such as those described in Current Protocols in Cytometry (John Wiley & Sons, NY, N.Y., Eds. J. Paul Robinson, et al.). Generally, a biological sample, such as peripheral blood, bone marrow, lymph node tissue, cord blood, thymus tissue, tissue from a site of infection, spleen tissue, tumor tissue, and the like, is collected from a subject and cells are isolated therefrom using techniques known in the art. In one embodiment, blood is collected from a subject and any mature erythrocytes are lysed using a buffer, such as buffered NH₄Cl. The remaining leukocytes are washed and then incubated with antibodies (e.g., monoclonal antibodies) conjugated to any of a variety of dyes (fluorophores) known in the art (see, for example, http://www.glenspectra.co.uk/glen/filters/fffluorpn.htm or http://cellscience.bio-rad.com/fluorescence/fluorophoradata.htm). Representative dyes in this context include, but are not limited to, FITC (Fluorescein Isothiocyante), R-phycoerythrin (PE), Allophycocyanin (APC), Cy7200 ), and Texas Red.

A wide variety of antibodies known in the art, and specific antibodies generated using techniques well known in the art, are useful in the context of the presently disclosed embodiments. Generally, the antibodies for use in the methods described herein are specific for a cell marker of interest, such as any of the CD cell surface markers (see, for example, the CD index at http://www.ncbi.nlm.nih.gov/PROW/guide/45277084.html; or Current Protocols in Immunology, John Wiley & Sons, NY, N.Y.), cytokines, adhesion proteins, developmental cell surface markers, tumor antigens, or other proteins expressed by a cell population of interest. An antibody specific for virtually any protein expressed by a cell is useful in the context of the present disclosure. Illustrative antibodies include antibodies that specifically bind to CD3, CD33, CD34, CD8, CD4, CD56, CD19, CD14, CD15, CD16, CD13, CD38, CD71, CD45, CD20, CD5, CD7, CD2, CD10 and TdT.

After a period of incubation with a dye-conjugated antibody, typically about 20 minutes in the dark (incubation times may vary according to particular protocols), the leukocytes are washed with buffered saline and resuspended in buffered saline containing protein for introduction into a flow cytometer.

The flow cytometer analyzes the heterogeneous cell population one cell at a time and can classify the cells based on the binding of the immunofluorescent monoclonal antibody and the light scattering properties of each cell (see, for example, Immunol Today 2000; 21(8): 383-90). Fluorescence detection is accomplished using photomultiplier tubes; the number of detectors (channels) determines the number of optical parameters the instrument can simultaneously examine while bandpass filters ensure that only the intended wavelengths are collected. Thus, flow cytometry can routinely detect multiple immunofluorescent markers in a quantitative manner and can measure other parameters such as forward light scatter (which is an indication of cell size) and right angle light scatter (which is an indication of cell granularity). Accordingly, a wide variety of cell populations can be differentiated and sorted using immunofluorescence and flow cytometry.

For example, by combining 4 colors of immunofluorescence with the physical parameters of forward light scatter (measure of cell size) and right angle light scatter (measure of cell granularity), a six dimensional data space can be generated wherein specific cell populations found in normal blood or bone marrow are restricted to small portions of the data space. As would be recognized by the skilled artisan after reviewing the specification, more or less than 4 colors of immunofluorescent markers could also be used. Excitation of fluoroflores is not limited to light in the visible spectrum; several dyes, such as the Indo series (for measuring intracellular calcium) and the Hoesch series (for cell-cycle analyses) are excitable in the ultraviolet range. Thus, some instruments currently available in the art are configured with ultraviolet-emitting sources, such as the four-laser, 10-color Becton Dickinson LSR II. Further, using a commercially available fluorescence activated cell sorter, such as the FACSVantage™ (Becton Dickinson, San Jose, Calif.), the EPICS® ALTRA® (Beckman Coulter, Fullerton, Calif.) or the MoFlo® sorter (DakoCytomation, Inc., Carpinteria, Calif.) cell populations can also be sorted into purified fractions.

In certain embodiments, staining for flow cytometric analysis is performed with an incubation at ambient temperature with titrated monoclonal antibodies (Mab) of interest followed by erythrocyte lysis using a ammonium chloride solution. In certain embodiments, cells are then fixed with 1% paraformaldehyde and analyzed on a flow cytometer, such as a BD FACS Calibur flow cytometer (Becton Dickinson, San Jose, Calif.). Note that where cells are sorted for further analysis, the cells are generally not fixed, but rather are sorted in a viable state. In certain embodiments, data analysis using gating on markers of interest is performed using WinList (Verity Software House, Topsham, ME). (Stelzer GT, et al., Ann N Y Acad Sci 1993; 677: 265-280.) Cell sorting on viable cells can be performed on any of a variety of cell sorters available, such as FACS Vantage SE cell sorter (Becton Dickinson) using selected antibody (e.g., monoclonal antibody) combinations to target the cell populations of interest.

Gene expression observed during the development of blood cells from hematopoietic stem cells to mature cells found in blood is a highly regulated process. (See Civin C I, Loken M R, Int'l J. Cell Cloning 1987; 5: 1-16.) Thus, specific, tightly controlled expression of genes occurs within not only different lineages of blood cells but also during different stages of maturation within those lineages. (See Loken, M R, et al., Flow Cytometry Characterization of Erythroid, Lymphoid and Monomyeloid Lineages in Normal Human Bone Marrow, in Flow Cytometry in Hematology, Laerum O D, Bjerksnes R. eds., Academic Press, New York 1992; pp. 31-42.) Not only do these gene products appear and/or disappear at precise stages of maturation, but the amounts of these glycoproteins are regulated within very tight limits in normal cells. It has been shown that these antigenic relationships are established early in fetal development and are constant throughout adult life on blood cells that are undergoing constant turnover and replenishment. (See LeBein T W, et al., Leukemia 1990; 4: 354-358.) These patterns and relationships of gene expression during maturation of normal cells are maintained following chemotherapy or even bone marrow transplantation. (See Wells D A, et al., J. Clin. Path. 1998; 110: 84-94.) Therefore, there is a very tightly coordinated regulation of multiple genes during normal development of blood cells both in terms of timing of expression as well as regulation of amounts of gene products expressed on the cell surfaces.

A comparison of normal antigen expression to neoplastic processes indicates that gene expression is disrupted in neoplastic cells. This gives rise to different antigenic relationships than those observed during normal maturation of cells. (See Hurwitz, C A, et al., Blood 1998; 72: 299-307.) These are not new antigens, but are those normally expressed gene products that have lost the coordinated regulation found in normal cells. Both acute lymphoblastic leukemia (“ALL”) and acute myeloblastic leukemia (“AML”) express antigens abnormally. (See Terstappen L W M M, Loken M R, Anal. Cell Path. 1990; 2: 229-240.) The types of abnormalities include lineage infidelity, defined as the expression of non-lineage antigens; antigenic asynchrony, e.g., the expression of antigens that normally appear on immature cells on mature cells; antigenic absence; and quantitative abnormalities. (See Terstappen L W M M, et al., Leukemia 1991; 6: 70-80.)

Not only are phenotypes of leukemic cells different from normal, the relationships between antigens are different from one case to the next, suggesting that each leukemic transformation causes a loss of coordinated gene regulation resulting in a unique phenotypic pattern for each leukemia. In 120 pediatric ALL cases and 86 adult AML cases each detailed phenotype was different from normal and from each other. (See Id.; Hurwitz, C A supra and Terstappen, supra) Thus, neoplastic transformation affects primary DNA sequencing (genotype) and the regulation of normal genes so that they are inappropriately expressed at the wrong time during development, expressed in the wrong amounts, and/or are expressed in context with other genes that are not observed in normal cells (phenotype). The loss of coordinated gene regulation appears to be a hallmark of neoplastic transformation that results in abnormal phenotypes where each leukemic clone is different from normal and is different from other leukemias of the same type.

It should be noted that embodiments are not limited to the analysis of leukemic cells. Embodiments can be applied to analysis of any of a variety of malignancies and other diseases including lymphoma of both the T and B cell type where abnormal cell types can be discerned by expression of cell surface markers or intracellular markers that can be detected by flow cytometry (e.g., acute lymphoblastic leukemia, chronic lymphocytic leukemia, hairy cell leukemia, lymphoma and myeloma).

Flow cytometry can be adopted to use this phenotypic difference from normal to aid in the detection and diagnosis of leukemia as well as in monitoring response to therapy. Flow cytometry has been used in hematopathology to phenotype the tumor, e.g., differentiating AML from ALL. The focus on neoplastic cells can extend to minimal disease detection, such as residual disease detection. However, conventional residual disease detection techniques employing flow cytometry and molecular techniques require a patient specific reagent panel to identify the specific phenotype observed at diagnosis. (See Reading C I, et al., Blood 1993; 81: 3083-3090.) Such patient specific panels have been used to detect residual ALL and AML down to levels of 0.03-0.05%. (See Coustan-Smith E, et al., Blood 2001; 96: 2691-2696; San Miguel J F, et al., Blood 2002; 98: 1746-1751.)

Conventional detection of residual disease using patient specific reagent panels, however, suffers from the following limitations: A diagnostic specimen with an aberrant phenotype is required in order to construct a panel. In 25% of cases an aberrant phenotype may not be identifiable. (See Vidriales, supra.) Processing time is substantial because a technician must examine prior analysis for the particular patient in order to determine the reagent combination to use in each case; The phenotype of a leukemic cell population that is different than the originally diagnosed phenotype may not be detected. For example, the phenotype may change from diagnosis to relapse as a result of clonal evolution or an outgrowth of a minor chemotherapy resistant subclone. (See San Miguel, supra.) Unexpected or unanticipated abnormalities, such as secondary myelodysplasia or abnormalities in other lineages may be overlooked.

The assessment of residual disease using patient specific panels can work well in a controlled environment, such as a research study where there is access to all sequential specimens and there is high compliance in obtaining specimens at specific times in therapy. In clinical practice, however, a flow cytometry laboratory may be asked to perform residual disease analysis when the laboratory did not perform the initial diagnosis. A detailed immunophenotype is often unavailable or incomplete.

Minimal disease detection can also be performed using standardized panels and difference from normal as the tumor specific marker (See Wells D A, et al., Leukemia 1998; 12: 2015-2023; Sievers, et al., supra). In certain embodiments, molecular confirmation following flow cytometry is used. Coordinated gene expression is so precise that a divergence of ½ a decade in antigen expression is sufficient for the discrimination between normal and aberrant neoplastic cells. In such an approach, specific reagent panels are used for each suspected lineage, for example, B lineage ALL; AML; B and T lineage non-Hodgkins lymphoma (“B-NHL”, “T-NHL”), chronic lymphocytic leukemia, hairy cell leukemia, myeloma and T lineage ALL (“T-ALL”). Tumor populations can be identified by first identifying patterns expected of normal cells, then focusing on cells that do not match the patterns expected of normal cells.

For example: In hematopoietic stem cell transplants for ALL, flow cytometry was shown to be more sensitive and more specific than morphology, cytogenetics, or the two technologies combined, in predicting relapse for 120 patients (See Wells D A, supra.); In pediatric AML flow cytometric detection of residual disease was the best predictor of outcome in 252 patients studied. (Sievers E L, etal., Blood 2003; 101: 3398-3406.) Patients with detectable tumor at any time during therapy were 4 times more likely to relapse and 3 times more likely to die than those patients in whom no tumor was detected; In hematopoietic stem cell transplants flow cytometry is able to distinguish between normal regenerating blasts and recurrent tumor based on aberrant antigen expression. (See Shulman H, et al., Am J Clin Path 1999; 112: 513-523.) Patients can exhibit 20% normal blasts in the blood or may have up to 50% regenerating blasts in the marrow without detection of neoplastic cells.

The detection of abnormal phenotypes of small populations of cells in blood or bone marrow extends the utility of flow cytometry to other applications beyond simply phenotyping leukemias. Flow cytomery has been used to show that a significant proportion (10%) of patients with a diagnosis of myelodysplasia have been misdiagnosed and have lymphoid, not myeloid abnormalities.

There are several advantages of minimal disease detection based on difference from normal. For example, the technique does not require a diagnostic specimen for creation of a specific panel; the approach allows for rapid processing of specimens in a high volume laboratory with identical panels being used for different patients; the results are not affected by a change in phenotype following therapy; and proper standardized panel selection permits the detection of unexpected or unanticipated findings that are the result of hematologic abnormalities.

Once the abnormal cells are identified by flow cytometry as described herein, the cells are sorted and collected for further confirmatory genetic analysis. A desired number of cells is collected using the parameters of the flow cytometer according to established protocols known to the skilled artisan and as described in the art, for example, in Current Protocols in Cytometry (John Wiley & Sons, NY, N.Y., Eds. J. Paul Robinson, et aL.). The cells are collected into one or more drops of collection fluid. In one embodiment, single cells are collected in each small drop of collection fluid. As the cells are collected, the drops containing the desired sorted, purified population of cells (fraction) are deposited into tubes, plates, or onto a solid support (see, e.g., U.S. patent application Ser. No. 11/096,207). Many distinct populations of cells can be sorted, as defined by immunofluorescent markers as described herein. Accordingly, 1, 2, 3, 4, 5, 6, or more distinct populations (fractions) of cells can be sorted and collected as described herein. In certain embodiments, the sorted cells comprise B cells, T cells, NK cells, plasma cells, stem cells, granulocytes, basophils, or other cells found in the blood.

One advantage of the present invention is that very few cells are needed for genetic analysis to confirm the presence of minimal disease. Thus, in certain embodiments, the number of sorted cells to be analyzed can be about 2000,1500, 1000, 950, 900, 850, 800, 750, 700, 650, 600, 550, 500, 450, 400, 350, 300, 250, 200 or fewer cells.

Genetic Analysis of Sorted Cells

Following detection of minimal disease using flow cytometry fluorescence activated cell sorting, the sorted cells are subjected to genetic analysis using any of a variety of reagents and techniques known in the art and described for example, in Current Protocols in Molecular Biology (John Wiley & Sons, NY, N.Y.), or Innis, Ed., PCR Protocols, Academic Press (1990). In this regard, any genetic analysis that confirms that the sorted population of cells represents minimal disease is contemplated for use herein. The genetic analysis to be performed will depend on the disease setting and can be determined by the skilled artisan. One advantage of the present invention is that the confirmatory genetic analysis does not require patient-specific reagents. However, such reagents can be used where available if desired.

Nucleic acid from the sorted cells is isolated using techniques known in the art such as described in Current Protocols in Molecular Biology (John Wiley & Sons, NY, N.Y.) or using any of a variety of commercially available reagents. The nucleic acid for subsequent analysis may be genomic DNA, RNA, including HnRNA and mRNA, or cDNA.

Examples of subsequent analysis which may be performed on samples of genetic material isolated from the sorted cell population of interest include, but are not limited to, polymerase chain reaction (PCR), ligase chain reaction (LCR), reverse transcriptase initiated PCR (RT-PCR), DNA or RNA hybridization techniques including restriction fragment length polymorphism (RFLP) and other techniques using genetic probes such as fluorescence in situ hybridization (FISH), DNA analysis by variable number of tandem repeats (VNTR) or short tandem repeats (STR), or other genotype analysis, CpG methylation analysis (see, for example, Cottrell et al., Nucleic Acids Research 2004; Vol. 32, No. 1 e10), genomic sequencing, enzymatic assays, affinity labeling, methods of detection using labels or antibodies and other similar methods.

The genetic analysis may involve the use of oligonucleotides. As used herein oligonucleotides is used as the term is normally understood in the art, that is, to mean a short string of nucleotides. In this regard, the oligonucleotides can be used as either primers or probes and can be of varying lengths as is appropriate for the molecular technique they are being used for, such as PCR, RT-PCR, hybridization assays, FISH, and the like. Generally oligonucleotides are from about 8-50 nucleotides in length but they can be shorter or much longer. In some embodiments, the oligonucleotides can be 10,11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more nucleotides in length. In certain embodiments, the oligonucleotides can be 65, 70, 75, 80, 85, 90, 95, 100, 105,110, 115,120, 130, 140, 150, or even 200 nucleotides in length. As would be recognized by the skilled artisan, oligonucleotides can be synthesized or otherwise constructed using techniques well known in the art.

In one embodiment, the genetic analysis comprises the detection of a translocation event. Illustrative translocation events include, but are not limited to: BCR-ABL; BCL1/JH t(11;14); BCL2/JH t(14;18); BCR/ABL t(9;22); PML/RAR t(15;17); translocations involving MLL and any of its translocation partners (e.g., AF4, AF6, AF9, ENL and ELL), the t(10;11)(p12;q23) translocation, which is a recurrent event in acute myeloid leukemias; c-myc (8q24) translocations involving t(8;14) (translocations involving t(8;14) occurs in less than 5% of human multiple myeloma cases, but between 10 to 20% of tumors have genetic abnormalities near this locus (Bergsagel, 1998); Bcl-1/PRAD-1/cyclin D1 (11q13). A cluster of translocation events occur in this locus resulting in chronic lymphocytic leukemia (CLL) and lymphoma; FGFR3 and/or MMSET (4p16.3) Approximately 25% of myeloma has translocation of the receptor tyrosine kinase FGFR3 to the IgH locus; MUM1/IRF4/ICSAT/PIP/LSIRF (6p25) IRF4 is a member of the interferon regulatory factor family which are know to be involved in B-cell proliferation and differentiation. This recurrent translocation was seen in 2 of 11 MM; Cyclin D3 (6p21) is overexpressed in ˜3% of multiple myeloma cell lines and ˜4% of primary multiple myeloma tumors; c-maf (16q23) Translocation of c-maf into the IgH locus results in overexpression of c-maf in approximately 25% of multiple myeloma cell lines. And other translocation events known in the art. Translocation events can be detected using any of a variety of techniques known in the art, such as by FISH and PCR.

In certain embodiments, the genetic analysis comprises clonal B-cell or T-cell gene rearrangement detection (see, e.g., U.S. Pat. Nos. 5,296,351 and 5,418,134; see also U.S. Pat. No. 5,837,447). The immunoglobulin (Ig) and T-receptor (Tr) genes are present in all cells. Each consists of 4 families, the variable (V), diversity (D), joining (i), and constant (C) region family (except for immunoglobulin light chains which lack a D segment). In the process of development of a B- or T-lymphocyte, the gene is rearranged so that one randomly chosen member of each family is joined together to form the final molecule. Random mutations also occur at the VD, DJ and JC joining points. As a result of this random joining and mutation, the final immunoglobulin or T-receptor molecule is virtually unique. However, there are two features of particular importance with regard to the present invention: (a) The number of bases removed and/or inserted at the VD, DJ and JC junctions is quite variable, so that different immunoglobulin or T-receptor molecules, particularly the segments spanning VD, DJ and JC junctions, differ substantially in length. (b) There are some regions of high similarity if not of absolute identity within the genes. These comprise certain parts of the V regions, termed “framework” regions, and parts of the J and C regions.

PCR is then carried out utilizing consensus primers to regions which have a similar but not identical sequence in the immunoglobulin and T-receptor genes respectively. These regions comprise the framework portions of the V regions of the immunoglobulins, conserved V regions of the T-receptor genes, and parts of the D,J and/or C regions of the immunoglobulin or T-receptor genes. Generally, the primers will recognize and amplify only the final mature immunoglobulin or T-receptor molecule.

Use of such consensus primers in a PCR reaction results in amplification of the inter-primer segments of all mature immunoglobulin or T-receptor molecules in a tissue sample. The amplified segments will cross the VD, DJ (and JC) junctions. As a result the final amplified piece of DNA, irrespective of its sequence, will have a single length if all of the immunoglobulin or T-receptor genes in the tissue are derived from a monoclonal, malignant population or will have a heterogenous length if the molecules are derived from a heterogenous non-malignant population. Primers for the immunoglobulin molecule will identify malignant populations of B-lymphocyte origin, primers for the T-receptor molecule will identify malignancy of T-lymphocyte origin.

The lengths of the amplified pieces of DNA can be simply determined by separating the DNA molecules by a technique which separates molecules on the basis of size such as electrophoresis in agarose or polyacrylamide gel, or chromatography. In certain embodiments, PCR amplicons are analyzed by differential fluorescence detection using the ABI310 capillary electrophoresis sequencer and the ABI Prism® GeneScan® Analysis software.

Illustrative primer sets for detection of clonal B-cell rearrangement include the Biomed-2 primer sets for the Immunoglobulin Heavy chain region of framework 1, 2 and 3 (see, e.g., van Dongen J J, et al., Leukemia 2003; 17: 2257-317). Such primers can be synthesized using techniques known in the art and are also commercially available (see, e.g., InVivoScribe Technologies, San Diego, Cat# 1-101-0021). Illustrative primer sets for detection of clonal T cell receptor gamma gene rearrangement assay include commercially available primer sets (see, e.g., InVivoScribe Technologies, San Diego, Cat# 1-207-0011). As would be recognized by the skilled artisan, primers for use as described herein can be designed using immunoglobulin and T cell receptor sequences available in the art and any of a variety of primer design computer software programs, such as free programs (http://www.rfcgr.mrc.ac.uk/GenomeWeb/nuc-primer.html) and commercially available programs.

Thus the present invention provides methods for monitoring, staging and diagnosis in a variety of diseases including any of a number of cancers. Illustrative cancers where the present invention is useful include, multiple myeloma, plasmacytoma, macroglobulinemia, acute lymphoblastic leukemia (ALL), acute myelogenous leukemia (AML), chronic myelogenous leukemia (CML), chronic lymphocytic leukemia (CLL), lymphomas, any other cancer involving T cells or B cells. In certain embodiments, a patient may be afflicted with one or more of the above cancers, such as myeloma and CLL. In this regard, the present invention can be used to determine whether the two cancers arose from two separate events or whether they are actually the same disease (e.g., a lymphoplasma cytoid). In such a setting, the abnormal myeloma and lymphoid cells are sorted and the molecular clonal signatures of the two populations can be compared. If the signatures are different, then two, independent events gave rise to the two cancers. If the signatures are the same, then the two cancers are the same.

In another embodiment, true remission in myeloma can be determined by sorting plasma cells (e.g., antibody-producing B cells) and carrying out molecular clonal analysis as described herein on this sorted population. Without sorting, the results are confounded by large numbers of B cells. However, by first sorting the plasma cells which contain suspected aberrant cells (˜0.2% of B cell population), the aberrant cells represent a larger percentage of the cells analyzed and the neoplastic clonal population can more easily be detected by molecular techniques.

The present invention is particularly useful in settings where very low numbers of abnormal lymphoid populations are present. B cell gene rearrangement PCR alone can be used to assess and monitor clonality if the suspected malignant population is present at a level of approximately 1 %. The use of allele-specific (ASO) primers in combination with germline (Jg) primers and (Jg) TaqMan probes have been shown as a useful tool with a sensitivity of 0.01 (10e-4) to a maximum of 0.001% (10e-5). (van der Velden V H, et al., Leukemia 2003; 17:1013-34; van derVelden V H, et al., Leukemia 2002; 16: 1372-80; van der Velden V H, et al., Leukemia 2002; 16: 928-36; Pongers-Willemse M J, et al., Leukemia 1998; 12: 2006-14; Bruggemann M, et al., Leukemia 2000; 14: 1419-25; Donovan J W, et al., Blood 2000; 95: 2651-8.) However, in many cases those sensitivities cannot be reached due to non-specific amplification of gene rearrangements in the normal lymphocytes present. After treatment, the background of normal B and T cells may be particularly high, lowering the sensitivity even further. Furthermore, the design and optimization of patient-specific assays may be too time-consuming or expensive to allow a broad clinical application. Thus, the present invention can be used to assess and monitor clonality where the suspected B cell malignant population is present at a level of less than about 1% of nucleated cells from the patient sample without the use of patient-specific reagents. Patient samples can be from blood, leukaphersis, biopsy, bone marrow, tissues, body fluid and the like.

The interpretation of putative monoclonal peaks can be particularly difficult in specimens with small T cell populations. Whereas the detection levels for TCRG gene rearrangement analysis have been reported at 1% to 0.1% [10e-2 to 10e-3] (Delabesse E, et al., Leukemia 2000; 14: 1143-52), the sensitivity of the TCRG PCR assay is highly variable from patient to patient, dependent upon the specific gene rearrangement's polyclonal background. The TCRG gene rearrangement assay can indeed detect tumor cells at 1%, but for a subset of tumor specimens strong monoclonal signatures are achieved only if the abnormal cells are present at 5 to 10% or greater. Moreover, if the size of the patient specific gene rearrangement signal is unknown, the interpretation of small putative monoclonal peaks can be difficult. These findings are in agreement with the literature reporting that the sensitivity of T cell receptor genes as PCR targets is dependent upon the frequency of comparable gene rearrangements in normal cells (van Wering E R, et al., Leukemia 2001; 15: 1301-3). In particular, TCR gamma (TCRG) gene rearrangements are known for their limited sensitivity due to the limited size of TCRG junctional regions and the abundant background of polyclonal TCRG gene rearrangements in normal T cells (van der Velden V H, et al., Leukemia 2003; 17: 1013-34; van Wering E R, et al., Leukemia 2001; 15 :1301-3). TCRG gene rearrangements occur on both alleles in virtually all CD3+lymphocytes and show limited combinatorial and junctional diversity. On the other hand TCRG gene rearrangements are known for their high stability from diagnosis to relapse in T-ALL since they are mostly end-stage rearrangements. (Szczepanski T, et al., Leukemia 2003; 17: 2149-56.) Over-interpretation of small suggestive peaks must be avoided since oligoclonal/clonal T cell expansions can also be found in healthy individuals. So-called ‘canonical’ TCRG rearrangements resulting from accumulation of TCRGγδ+ T-lymphocytes can particularly be found in peripheral blood and increase in frequency with age. (van Dongen J J, et al., Leukemia 2003; 17: 2257-317; Posnett D N, et al., J Exp Med 1994; 179: 609-18; Hodges E, et al., J Clin Pathol 2003; 56: 1-11.) Reactive oligoclonal populations can also occur following transplantation that represent re-population of the bone marrow, and which cannot be interpreted as re-occurrence of malignancy without reference to the original clone. Therefore, the invention described herein is useful to link abnormal phenotype with monoclonality and the converse. Thus, the present invention can be used to assess and monitor clonality where the suspected T cell malignant population is present at a level of less than about 1% of nucleated cells without the use of patient-specific reagents.

In certain embodiments the present invention can be used to assess and monitor clonality where the suspected malignant B or T cell population is present at a level of less than about 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002% , or 0.001% and lower, without the use of patient-specific reagents. In a further embodiment, the present invention can be used to assess and monitor clonality where the suspected malignant B or T cell population is present at a level of between about 0.9% and about 0.001% without the use of patient-specific reagents. In another embodiment, the present can be used to assess and monitor clonality where the suspected malignant B or T cell population is present at a level of between about 0.8% and about 0.005%, between about 0.7% and about 0.005%, between about 0.6% and about 0.005%, between about 0.5% and about 0.005%, between about 0.5% and about 0.01%, or between about 0.5% and about 0.001%, without the use of patient-specific reagents.

The following Examples are offered by way of illustration and not by way of limitation.

### EXAMPLES

### Example 1

### Cell Sorting and Genotype Analysis of Abonrmal B and T Lymphocytes

This example describes a quantitative two-step technique for detecting and confirming low levels of disease by integrating phenotype analysis using standardized flow cytometry panels, cell sorting and genotype analysis using multiplex gene rearrangement PCR thereby confirming the presence of both aberrant phenotype linked to a specific genotype. The feasibility of combining cell sorting with clonality profiling to effectively lower sensitivity limits for disease detection and to provide independent confirmation of the tumor detection without the need for patient specific assay designs is demonstrated. Bone marrow aspirates from three patients with small abnormal B lymphoid populations and one peripheral blood specimen with a small aberrant T cell population as detected by flow cytometry were analyzed by Immunoglobulin heavy chain (IgH) or T cell receptor gamma chain (TCRG) gene rearrangement PCR with and without cell purification to illustrate the utility of relating aberrant phenotype to a specific genotype for several clinical applications.

Materials and Methods

Patient Information

Peripheral blood or bone marrow from 4 patients with low percentages of abnormal T- or B-lymphoid populations as determined by flow cytometric analysis were analyzed by gene rearrangement detection combined with flow cell sorting.

Flow Cytometry and Cell Sorting

Antibody combinations, their sources and the detailed procedures for flow cytometric analysis have been described previously. (Wells D A, et al., Leukemia 1998; 12: 2015-23.) Briefly, staining for flow cytometric analysis was performed with a 20 min incubation at ambient temperature with titrated monoclonal antibodies (Mab) of interest followed by erythrocyte lysis using a ammonium chloride solution. Cells were then fixed with 1% paraformaldehyde and analyzed on a BD FACS Calibur flow cytometer (Becton Dickinson, San Jose, Calif.). Data analysis using CD45 gating was performed using WinList (Verity Software House, Topsham, Me.). (Steizer G T, et al., Ann N Y Acad Sci 1993; 677: 265-280.) Cell sorting on viable cells was performed on a FACS Vantage SE cell sorter (Becton Dickinson) with selected Mab combinations to target the cell populations of interest.

PCR Studies of Gene Rearrangements

Genomic DNA was isolated from sorted and unsorted cell specimens using the QIAamp® DNA Mini Kit (Qiagen, Cat# 51306) according to the manufacturer's instructions. For clonal B-cell gene rearrangement detection DNA was amplified using the Biomed-2 (van Dongen J J, et al., Leukemia 2003; 17: 2257-317) primer sets for the Immunoglobulin Heavy chain region of framework 1, 2 and 3 according to the manufacturer's instructions (InVivoScribe Technologies, San Diego, Cat# 1-101-0021). To identify T cell clonality the T cell receptor gamma gene rearrangement assay (InVivoScribe Technologies, San Diego, Cat# 1-207-0011) was used according to the instructions. PCR amplicons were analyzed by differential fluorescence detection using the ABI310 capillary electrophoresis sequencer and the ABI Prism® GeneScan® Analysis software.

Staging Lymphoma

A staging bone marrow to assess the dissemination of lymphoma is a common application of minimal disease detection using flow cytometry. Often the diagnosis is made on a lymph node biopsy that is never sent for confirmation by flow cytometry. The quandary is detecting a small population of abnormal cells by flow cytometry and correlating these results with the morphologic diagnosis. Without a phenotypic fingerprint, the detection of abnormal cells is based on difference from normal. (Wells D A, et al., Am J Clin Pathol 1998; 110: 84-94; Sievers E L, et al., J Nat Can Inst 1996; 88: 1483-1488.) The specificity and sensitivity of the assay depends upon the reagents, instrument sensitivity, the frequency of the abnormal cells, the frequency of the normal counterparts and the relative differences between the normal and abnormal cells. All of these factors interact to raise or lower the level of confidence in concluding that the specimen contains tumor. This is especially critical when treatment will be based on the results of the test.

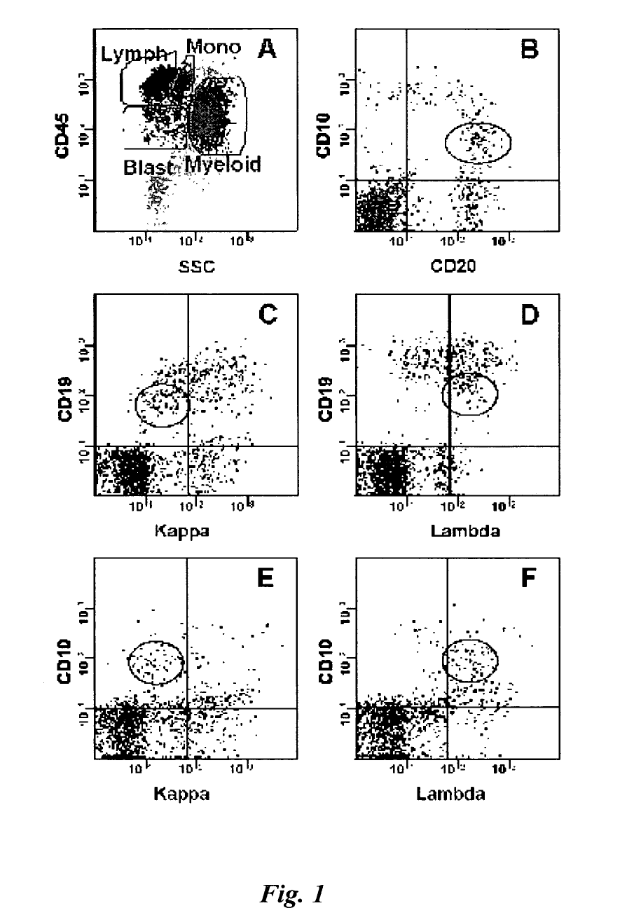

An example of this situation is seen when a bone marrow specimen from a 53 year old male recently diagnosed with follicular lymphoma was submitted for staging by flow cytometry. The original diagnosis was based on morphology and immuno-cytochemistry on a lymph node biopsy. An original diagnostic specimen was not available to assess tumor phenotype or genotype. The flow cytometry antibody panel including CD45, HLA-DR, CD19, FMC-7, CD20, CD10, CD5, CD22, CD25, CD23, and immunoglobulin light chains, kappa and lambda, showed slight increase in CD10 and CD20 staining intensity in cells phenotypically identical to Stage III immature B lymphoid cells [FIG. 1B]. (Loken M R, Wells D A, Normal antigen expression in hematopoiesis: Basis for interpreting leukemia phenotypes, in “Immunophenotyping”, Stewart C, Nicholson J. Eds. Wiley Liss, Inc. New York, 2000; 133-160.) A suggestive increase in lambda staining was detected in a small abnormal lymphoid population (0.6%) with dim CD19 expression [FIG. 1C,D]. Reprocessing the specimen correlating CD10 with surface kappa and lambda also suggested an increase in lambda staining, however this difference was not definitive since a majority of the B cell population (4.6% of non-erythroid cells) was polyclonal.

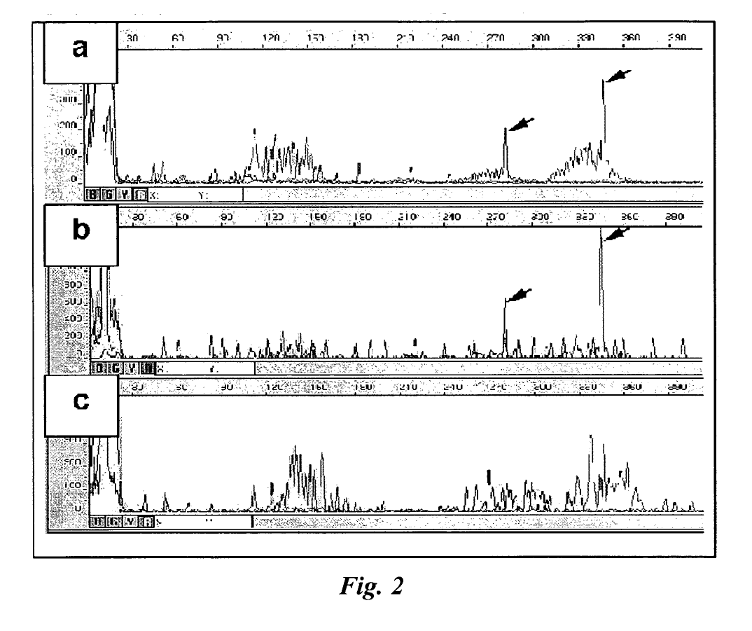

Therefore, B cell gene rearrangement studies were used in combination with flow cytometry based cell sorting to confirm monoclonality on that specific population of cells. Genomic DNA was extracted from 40,000 cells purified by flow cytometry for CD10 and bright CD45 expression and from 45,000 purified bright CD45 positive lymphocytes that did not express CD10. The isolated DNA was studied by genotype analysis using multiplex gene rearrangement PCR. Monoclonal peaks were detected at 346 bp for immunoglobulin heavy chain framework region one and at 281 bp for framework region two in the unsorted and in the CD10 positive sorted cell fraction (FIG. 2a, b). The sorted control cell fraction (CD10−/CD45+) containing the normal developing B cells and T cells showed polyclonal amplicon distribution for all three immunoglobulin framework regions (FIG. 2c). These data demonstrate that the abnormality detected by flow cytometry and by gene rearrangement studies were identifiable in the same cell population. Cell purification allows for independent phenotype and genotype studies to be performed on exactly the same aberrant cells, not just correlative on the entire specimen, thus increasing the specificity of both techniques.

Residual Disease Monitoring

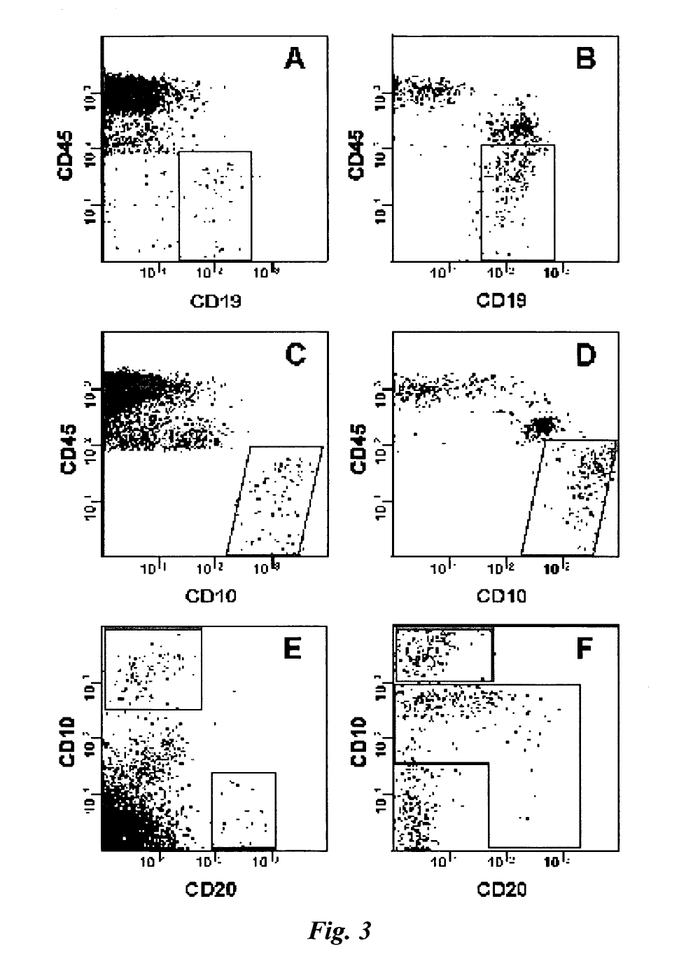

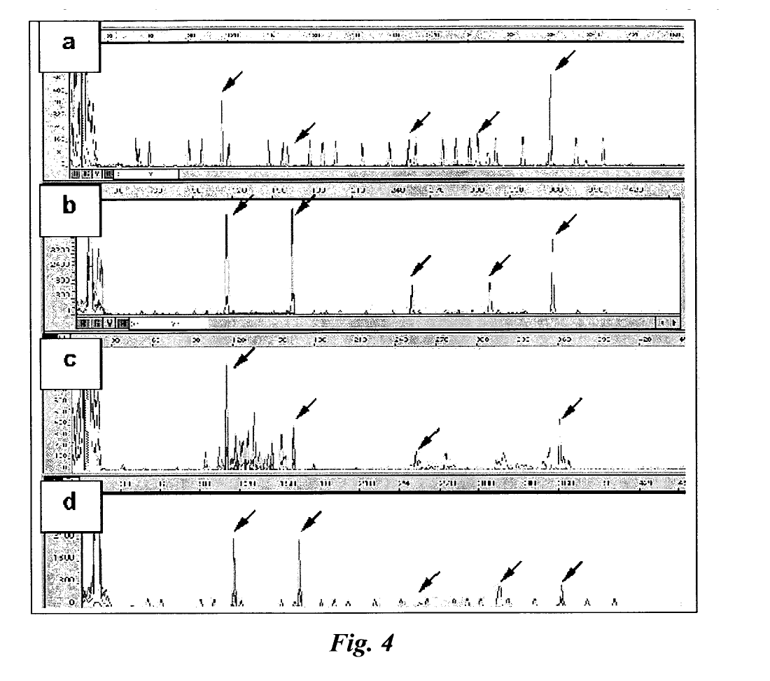

Detection of low levels of tumor cells following therapy is the most frequent use of minimal disease detection. Standardized panels of monoclonal antibodies can be used for residual disease detection circumventing the requirement for a diagnostic specimen or the detection of a clonal population that changes phenotype. (Wells D A, et al., Am J Clin Pathol 1998; 110: 84-94; Sievers EL, et al., J Nat Can Inst 1996; 88: 1483-1488.) This approach is useful in a bone marrow transplant setting where the patient is often first encountered when in remission. The detection of minimal residual disease is illustrated in a specimen from a 19 year old patient post-hematopoietic stem cell transplant for precursor B-lineage acute lymphoblastic leukemia (B-ALL). The patient had relapsed post transplant and was given chemotherapy to induce remission. A bone marrow aspirate was obtained to assess remission status. Upon analysis of the bone marrow 0.05 % abnormal lymphoblasts were detected by flow cytometry, expressing HLA-DR, bright CD10, dim CD19, heterogeneous bright CD34, but lacking expression of CD45 (FIG. 3A, C, E). No monoclonal or polyclonal signal was detected by the IgH gene rearrangement assay in this bone marrow specimen due to the presence of very few normal B cells (0.02%) (FIG. 3E). The small abnormal lymphoblast population was sorted using a CD10+and CD45−gate (FIG. 3C) and 800 purified cells were analyzed for B cell clonality by PCR. The sorted tumor cell population had a monoclonal peak profile with amplicons for all three IgH framework regions (FIG. 4a). The combination of aberrant phenotype and monoclonal cell population demonstrated that the tumor was still detectable at 0.05%.

A follow-up bone specimen was received 5 weeks later and flow cytometry revealed increased abnormal lymphoblasts at 4.8% with an identical phenotype as detected before, clearly indicating relapse (FIG. 3B, D, F). B cell gene rearrangement analysis of this unseparated bone marrow specimen resulted in monoclonal amplicons identical in size to the clonality profile detected in the previous sorted cell fraction (FIG. 4b). After further chemotherapy, an additional bone marrow aspirate obtained 4 weeks later, contained 0.3% residual abnormal lymphoblasts by flow cytometry analysis. B cell gene rearrangement analysis of the unseparated bone marrow did not result in a distinct monoclonal profile due to the low percentage of tumor cells (FIG. 4c). However, the B cell clonality profile of the CD10 positive sorted cell population again revealed clonal peak sizes identical to previous results (FIG. 4d).

B cell gene rearrangement PCR alone can be used to assess and monitor clonality if the suspected malignant population is present at a level of approximately 1%. To increase sensitivity for minimal residual disease monitoring, the use of allele-specific (ASO) primers in combination with germline (Jg) primers and (Jg) TaqMan probes have been shown as a useful tool with a sensitivity of 0.01 (10e-4) to a maximum of 0.001% (10e-5). (van der Velden V H, et al., Leukemia 2003; 17: 1013-34; van der Velden V H, et al., Leukemia 2002; 16: 1372-80; van der Velden V H, et al., Leukemia 2002; 16: 928-36; Pongers-Willemse M J, et al., Leukemia 1998; 12: 2006-14; Bruggemann M, et al., Leukemia 2000; 14: 1419-25; Donovan J W, et al., Blood 2000; 95: 2651-8.) However, in many cases those sensitivities can not be reached due to non-specific amplification of gene rearrangements in the normal lymphocytes present. After treatment, the background of normal B and T cells may be particularly high, lowering the sensitivity even further. Furthermore, the design and optimization of patient specific assays may be too time-consuming or expensive to allow a broad clinical application.

We have combined routine immunophenotyping with cell purification and subsequent gene rearrangement studies by multifluorescent PCR and capillary electrophoresis analysis to confirm the presence of a monoclonal leukemic cell population and to lower the assay sensitivity level. Suspicious cell populations down to levels of 0.01% can be identified and purified by flow cytometry. Subsequent gene rearrangement analysis can confirm the identity of putative monoclonal peaks in reference to the unsorted specimen and/or to the original clone from a diagnostic specimen or paraffin-embedded tumor biopsy. The combination of the two technologies allows the identification of a clonogenic neoplastic cell population which would be undetectable or inconclusive by conventional analysis. For minimal disease monitoring the amplicon size of the clonal gene rearrangement, known from a previous diagnostic marrow aspirate or paraffin-embedded biopsy specimen, becomes the tumor specific marker without the need to develop patient specific DNA primer, probes or antibody panels. This approach could also be applicable to demonstrate that a suspicious phenotype is not monoclonal and/or recurrent disease, thus preventing potential additional chemotherapy for the patient.

Initial Diagnosis

In addition to minimal residual disease monitoring, cell sorting in combination with clonality profiling can also provide valuable confirmatory data in primary diagnostic specimens with low proportions of neoplastic cells. Patients presenting with anemia and/or pancytopenia often provide a difficult diagnostic dilemma. In addition to multiple non neoplastic etiologies, clonal processes in myeloid, T or B cells can cause suppression of hematopoiesis. The abnormal cell population may constitute a minor proportion of the specimen yet can influence the production of cells of multiple lineages.

B-cell

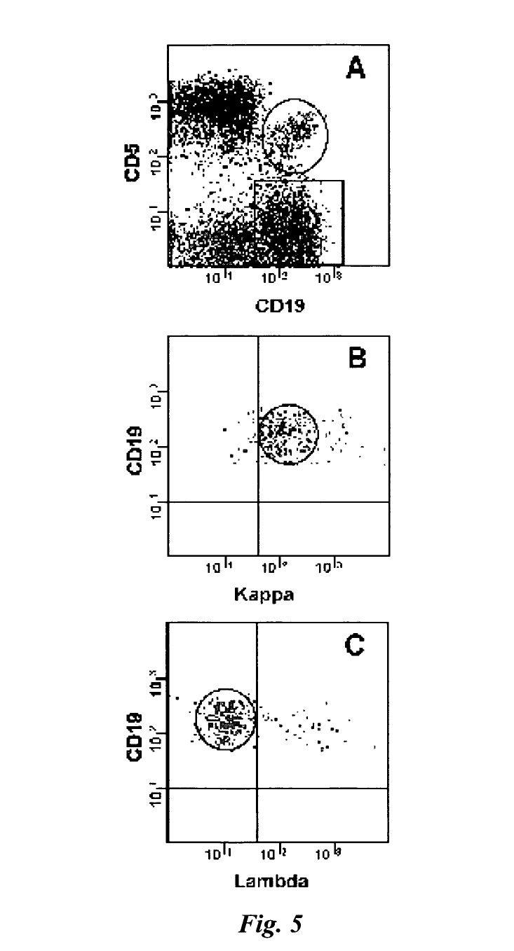

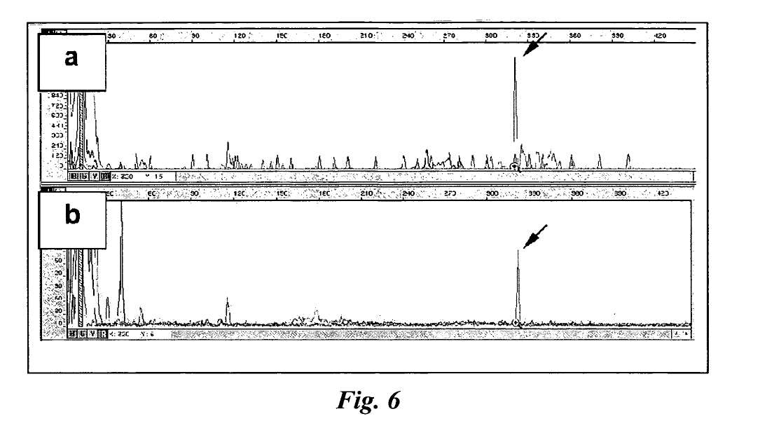

A diagnostic bone marrow aspirate was obtained from a 66 year old female with a listed history of autoimmune hemolytic anemia and cold agglutinin. Flow cytometry findings revealed a small, abnormal B-lymphoid population at 0.6% of the total non-erythroid cells positive for HLA-DR, CD38, CD19 and bright CD20 (FIG. 5). Normal B lymphoid cells were more frequent at 6.1% of the non-erythroid cells. Four color flow cytometric analysis of CD5, CD19, CD45 combined with immunoglobulin light chain expression demonstrated a predominance of surface kappa light chain immunoglobulin expressed on the CD19+/CD5+/bright CD45+ cells (FIG. 5 B,C). However, there still was a background of cells expressing lambda light chain. Since this was the primary diagnostic specimen for this patient, the identity of an aberrant B cell population required confirmation by other techniques. Gene rearrangement analysis of the bone marrow specimen detected a suspicious but not definitive monoclonal peak at 320 bp for the immunoglobulin heavy chain framework one region (FIG. 6a). Cells positive for CD5 and CD19 expression were sorted by flow cytometry with subsequent DNA extraction and analysis for B-cell gene rearrangements. A distinct monoclonal peak at 320 bp for FR1 was detected in the purified tumor cell fraction (FIG. 6b).

Since the specimen contained only a small number of abnormal cells (resulting in only 700 purified cells), it was important to rule out pseudoclonality. Pseudoclonality can be caused by the high sensitivity of PCR, amplifying gene rearrangements derived from a limited number of lymphoid cells present in the specimen, for example during or after chemotherapy treatment or in a fine needle aspirate. (van Dongen J J, et al., Leukemia 2003; 17: 2257-317.) Pseudoclonality can be ruled out either by comparing monoclonal peak sizes from sorted cell populations to a known tumor profile from a previous specimen or, alternatively, comparing the peak positions to suggestive monoclonal peaks in the unseparated sample in order to confirm identity of the monoclonal signature.

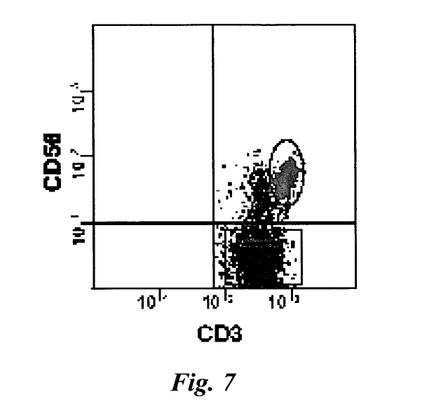

T-cell T cell gene rearrangement studies are less sensitive and specific in detecting small populations of cells as compared to B cell gene studies due to primer cross-reactivity with polyclonal background and the high frequency of benign clonal T cell expansions, particularly in peripheral blood. (van der Velden V H, et al., Leukemia 2002; 16: 1372-80; van Dongen J J, et al., Leukemia 2003; 17: 2257-317; van Wering E R, et al., Leukemia. 2001; 15: 1301-3; Szczepanski T, et al., Leukemia 2003; 17: 2149-56; Posnett D N, et al., J Exp Med 1994; 179: 609-18.) Cell purification based on phenotype can solidify the identification and characterization of abnormal T cell populations. A 72 year old female with a listed clinical history of anemia was shown to have 5 % abnormal T cells in the peripheral blood specimen by flow cytometry. The cells were small, and expressed increased CD3, reduced CD5, CD56 as well as normal levels of T cell antigens CD2, CD7, but did not express CD4, CD8, CD1a , CD25 or CD30 (FIG. 7). The abnormal cells exhibited TCR gamma/delta while the normal T cells expressed TCR alpha/beta.

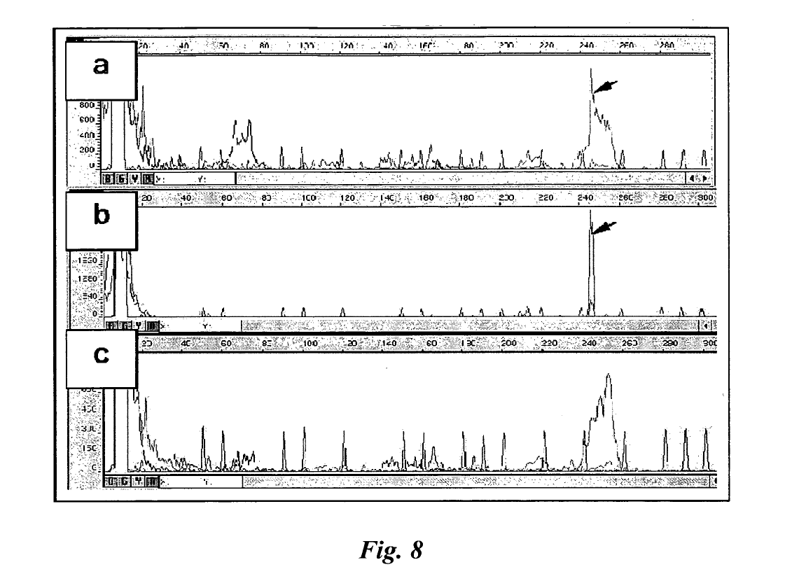

Total peripheral blood was analyzed by T cell gene rearrangement PCR and a putative monoclonal peak was detected at 245 bp (FIG. 8a). Using a CD56+ and CD3+ gate, 20.000 cells of the abnormal T cell population were purified for comparison to the normal T cells that did not express CD56 (FIG. 7). Subsequent analysis for T cell receptor gamma gene rearrangement demonstrated a single monoclonal peak at 245 bp matching the putative peak in the unsorted specimen (FIG. 8b). No distinct clonal peak was detected in the internal control cell fraction purified by a CD56 negative and CD3 positive gate (FIG. 8c).

Although the reactivity with discrete monoclonal antibodies is suggestive for the expansion of a particular granular lymphocyte population in LGL diagnosis, it is important to prove clonality. (Semenzato G, et al., Blood 1997; 89: 256-60.) Lymphoproliferative disease of granular lymphocytes is a heterogenous disorder resulting from the chronic proliferation of granular lymphocytes (GL). Clonal proliferations of these cells are considered as large granular lymphocytic leukemia (LGL) and diagnostic criteria have been defined in the past as evidence of granular lymphocytosis greater than 2,000/μL lasting for more than 6 months. In addition, pure red cell aplasia, characterized by anemia, is known to be associated with T-cell LGL leukemia. (Lacy M Q, et al., Blood 1996; 87: 3000-3006.) Updated criteria for LGL diagnosis call for the demonstration of an expansion of a restricted granular lymphocyte subset, even in patients with a relatively low GL count. (Semenzato G, et al., Blood 1997; 89: 256-60.) Whereas T-cell large granular lymphocyte leukemia is clinically indolent, severe neutropenia associated with the disease can be reversed by cyclosporine treatment. (Sood R, et al., Blood 1998; 91: 3372-8.)

The interpretation of putative monoclonal peaks can be particularly difficult in specimens with small T cell populations. Whereas the detection levels for TCRG gene rearrangement analysis have been reported at 1% to 0.1% [10e-2 to 10e-3] (Delabesse E, et al., Leukemia 2000; 14: 1143-52), in our experience the sensitivity of the TCRG PCR assay is highly variable from patient to patient, dependent upon the specific gene rearrangement's polyclonal background. The TCRG gene rearrangement assay can indeed detect tumor cells at 1% but for a subset of tumor specimens strong monoclonal signatures are achieved only if the abnormal cells are present at 5 to 10% or greater. Moreover, if the size of the patient specific gene rearrangement signal is unknown, the interpretation of small putative monoclonal peaks can be difficult. These findings are in agreement with the literature reporting that the sensitivity of T cell receptor genes as PCR targets is dependent upon the frequency of comparable gene rearrangements in normal cells. (van Wering E R, et al., Leukemia. 2001; 15: 1301-3.) In particular, TCR gamma (TCRG) gene rearrangements are known for their limited sensitivity due to the limited size of TCRG junctional regions and the abundant background of polyclonal TCRG gene rearrangements in normal T cells. (van der Velden V H, et al., Leukemia 2003; 17: 1013-34; van Wering E R, et al., Leukemia. 2001; 15: 1301-3.) TCRG gene rearrangements occur on both alleles in virtually all CD3+lymphocytes and show limited combinatorial and junctional diversity. On the other hand TCRG gene rearrangements are known for their high stability from diagnosis to relapse in T-ALL since they are mostly end-stage rearrangements. (Szczepanski T, et al., Leukemia 2003; 17: 2149-56.) Over-interpretation of small suggestive peaks must be avoided since oligoclonal/clonal T cell expansions can also be found in healthy individuals. So-called ‘canonical’ TCRG rearrangements resulting from accumulation of TCRGγδ+ T-lymphocytes can particularly be found in peripheral blood and increase in frequency with age. (van Dongen J J, et al., Leukemia 2003; 17: 2257-317; Posnett D N, et al., J Exp Med 1994; 179: 609-18; Hodges E, et al., J Clin Pathol 2003; 56: 1-11.) Reactive oligoclonal populations can also occur following transplantation that represent re-population of the bone marrow, and which cannot be interpreted as re-occurrence of malignancy without reference to the original clone. Therefore flow cytometry cell sorting can become a helpful asset to link abnormal phenotype with monoclonality and the converse.

In summary, this study demonstrates the application of standard flow cytometry panels to identify and to purify rare abnormal B and T cell populations for further molecular analysis and its usefulness in minimal disease confirmation for staging, monitoring and diagnostic settings. The data presented in this report outlines the clinical utility of standard B and T cell gene rearrangement analysis in flow cytometry sorted abnormal cells to confirm and identify rare tumor populations for monitoring, staging and diagnosis.

From the foregoing it will be appreciated that, although specific embodiments of the invention have been described herein for purposes of illustration, various modifications may be made without deviating from the spirit and scope of the invention. Accordingly, the invention is not limited except as by the appended claims.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Moreover, all numerical ranges utilized herein explicitly include all integer values within the range and selection of specific numerical values within the range is contemplated depending on the particular use.

What is claimed is:

1. A method for detecting the presence of minimal disease in a cancer patient, comprising,
identifying a population of abnormal cells by flow cytometry;
sorting the population of abnormal cells; and
contacting nucleic acid isolated from the sorted cells with one or more oligonucleotides, wherein the one or more oligonucleotides are not patient-specific, and wherein the contacting determines the presence of a neoplastic genetic marker; thereby detecting the presence of minimal disease.

2. The method of claim 1 wherein the step of identifying the population of abnormal cells by flow cytometry comprises measuring forward scatter and side scatter in combination with the fluorescence intensity of a combination of two or more cell surface markers selected from CD10, CD45, CD38, CD138, CD19, CD34, CD20, CD22, CD45, CD3, CD56, CD4, CD8, CD5, CD7, and CD2.

3. The method of claim 2 wherein the two or more cell surface markers comprise CD5 and CD19.

4. The method of claim 2 wherein the two or more cell surface markers comprise CD5 and CD8.

5. The method of claim 2 wherein the two or more cell surface markers comprise CD10 and CD20.

6. The method of claim 2 wherein the two or more cell surface markers comprise CD3 and CD56.

7. The method of claim 2 wherein the two or more cell surface markers comprise CD3 and CD4.

8. The method of claim 2 wherein the two or more cell surface markers comprise CD3 and CD8.

9. The method of claim 2 wherein the two or more cell surface markers comprise CD5 and CD7.

10. The method of claim 2 wherein the two or more cell surface markers comprise CD5 and CD3.

11. The method of claim 2 wherein the two or more cell surface markers comprise CD2 and CD7.

12. The method of claim 2 wherein the two or more cell surface markers comprise CD2 and CD3.

13. The method of claim 2 wherein the two or more cell surface markers comprise CD5 and CD2.

14. The method of claim 2 wherein the two or more cell surface markers comprise CD38 and CD56.

15. The method of claim 2 wherein the two or more cell surface markers comprise CD138 and CD38.

16. The method of claim 2 wherein the two or more cell surface markers comprise CD138 and CD19.

17. The method of claim 2 wherein the two or more cell surface markers comprise CD38 and CD19.

18. The method of claim 1 wherein the nucleic acid is contacted with at least two oligonucleotides in a polymerase chain reaction.

19. The method of claim 18 wherein the at least two oligonucleotides specifically amplify clonally rearranged immunoglobulin genes.

20. The method of claim 19 wherein the clonally rearranged immunologlobulin gene is selected from the group consisting of an Ig heavy chain rearrangement, an Ig kappa gene rearrangement, and an Ig lambda gene rearrangement.

21. The method of claim 18 wherein the at least two oligonucleotides specifically amplify clonally rearranged T cell receptor genes.

22. The method of claim 20 wherein the clonally rearranged T cell receptor genes are selected from the group consisting of a T cell receptor beta chain gene rearrangement, a T cell receptor delta chain gene rearrangement, and a T cell receptor gamma chain gene rearrangement.

23. The method of claim 1 wherein the neoplastic genetic marker is a clonally rearranged T cell receptor gene.

24. The method of claim 1 wherein the neoplastic genetic marker is a clonally rearranged immunoglobulin gene.

25. The method of claim 1 wherein the number of sorted cells is between about 200 and 1000.

26. The method of claim 1 wherein the presence of minimal disease in the cancer patient is confirmed in about 2 days.

27. The method of claim 1 wherein the presence of minimal disease in the cancer patient is confirmed in about 3 days.

28. The method of claim 1 wherein the presence of minimal disease in the cancer patient is confirmed in about 4 days.

29. The method of claim 1 wherein the nucleic acid is DNA.

30. The method of claim 1 wherein the nucleic acid is RNA.

31. The method of claim 1 wherein the minimal disease is minimal residual disease.

32. The method of claim 1 wherein the population of abnormal cells comprises neoplastic B cells present at between about 0.8% and 0.001% of nucleated cells.

33. The method of claim 1 wherein the population of abnormal cells comprises neoplastic T cells present at between about 0.8% and 0.001% of nucleated cells.

34. A method for detecting the presence of minimal disease in a cancer patient, comprising,
identifying a population of abnormal cells by flow cytometry;
sorting the population of abnormal cells; and
contacting nucleic acid isolated from the sorted cells with at least two oligonucleotides in a polymerase chain reaction, wherein the at least two oligonucleotides specifically amplify clonally rearranged immunoglobulin genes and are not patient-specific; and wherein the amplification of a clonal population confirms the presence of minimal disease.

35. A method for detecting the presence of minimal disease in a cancer patient, comprising,
identifying a population of abnormal cells by flow cytometry;
sorting the population of abnormal cells; and
contacting nucleic acid isolated from the sorted cells with at least two oligonucleotides in a polymerase chain reaction wherein the at least two oligonucleotides specifically amplify clonally rearranged T cell receptor genes and are not patient-specific; and wherein the amplification of a clonal population confirms the presence of minimal disease.

36. A method for detecting the presence or absence of minimal disease in a cancer patient, comprising,
identifying a population of cells suspected of containing abnormal cells by flow cytometry;
enriching the population of cells suspected of containing abnormal cells by sorting said population of cells; and
contacting nucleic acid isolated from the enriched, sorted cells with one or more oligonucleotides, wherein the one or more oligonucleotides are not patient-specific, and wherein the contacting determines the presence or absence of a neoplastic genetic marker; thereby detecting the presence or absence of minimal disease.

37. The method of claim 36 wherein the population of cells suspected of containing abnormal cells comprises plasma cells.

38. The method of claim 36 wherein the population of cells suspected of containing abnormal cells is sorted based on high expression of CD38.

39. The method of claim 37 wherein the neoplastic genetic marker is a clonally rearranged immunoglobulin gene.

40. The method of claim 37 wherein the nucleic acid is contacted with at least two oligonucleotides in a polymerase chain reaction.

41. The method of claim 40 wherein the at least two oligonucleotides specifically amplify clonally rearranged immunoglobulin genes.

42. The method of claim 41 wherein the clonally rearranged immunologlobulin gene is selected from the group consisting of an Ig heavy chain rearrangement, an Ig kappa gene rearrangement, and an Ig lambda gene rearrangement.
